(19) United States
(12) Patent Application Publication
(10) Pub. No.: US 2026/0283806 A1
(43) Pub. Date: Sep. 24, 2026

(54) DISTAL CENTRALIZER FOR HIP IMPLANT SYSTEM

(71) Applicant: GLOBUS MEDICAL, INC., AUDUBON, PA (US)

(72) Inventors: John Loiacono, Collegeville, PA (US); David Stumpo, Trappe, PA (US); Carly McGonagle, Philadelphia, PA (US); Demetrios Tsitsios, Broomall, PA (US); Joseph Capozzoli, Mount Laurel, NJ (US); Vishal Hegde, Baltimore, MD (US)

(21) Appl. No.: 19/685,973

(22) Filed: May 22, 2026

Related U.S. Application Data
(63) Continuation-in-part of application No. 18/929,681, filed on Oct. 29, 2024.

(51) Int. Cl. A61F 2/36; A61F 2/30

(52) U.S. Cl. CPC A61F 2/3676; A61F 2/30723; A61F 2/367

(57) ABSTRACT

A hip implant system includes a hip stem and a distal centralizer securable to a distal end of the hip stem. The distal centralizer may assume a variety of configurations to aid in aligning the overall hip stem axis with the axis of the femoral canal into which the hip stem is to be inserted. The hip implant system is useful in cemented hip stem procedures.

### CROSS-REFERENCE TO RELATED APPLICATION

This application is a continuation in part of U.S. patent application Ser. No. 18/929,681, filed Oct. 29, 2024, the contents of all of which are incorporated by reference herein in its entirety for all purposes.

### FIELD

The present disclosure relates to hip implant systems, generally, and to a distal centralizer for hip implants, more particularly.

### BACKGROUND

Hip arthroplasty, often called hip replacement, is a surgical procedure used to reconstruct and resurface a hip joint that has been damaged by disease or injury (e.g., arthritis or hip fracture). Total hip arthroplasty (THA) devices may replace both the acetabulum and the femoral head which comprise the hip joint, where the femur articulates relative to the acetabulum. To replace the hip joint, the hip arthroplasty may include a femoral implant (or “hip stem”) secured to the end of the femur and an acetabular implant secured to the acetabulum that forms a replacement articulating surface which interfaces with the femoral implant. The femoral implant is pivotably coupled to the acetabular implant, thereby reconstructing the hip joint.

During a total hip arthroplasty, a femoral neck osteotomy may be performed to remove the femoral head. The surgeon will then locate the femoral canal, and progressively broach the bone until the cortical walls of the femur have been contacted. After a trial reduction is conducted to ensure the correct femoral head and neck offset is chosen, the stem of the implant (“hip stem”) is inserted into the broached canal. In the case of a cementless implant, the femoral implant, after impaction, creates a press fit between the implant and the cortical walls of the femur.

When implanting a cemented hip stem, surgeons often use an implant to center the hip stem with the anatomic canal axis to prevent misalignment, prevent edge loading, and create a uniform cement mantle around the distal aspect of the hip stem.

Proper alignment and positioning is desirable to increase post-operative performance of the hip implant and reduce the risk of sub-optimal bio-mechanical conditions. on the placement of the stem such that the stem axis is in line with the anatomic canal axis.

It would thus be advantageous for a hip system to have features or components to facilitate proper alignment.

### SUMMARY

According to one implementation, a hip implant system is provided for use in an arthroplasty procedure for a patient having a femur with an anatomical axis. The system includes a hip stem extending from a base to a distal end and configured to be inserted into and secured within a femoral canal. The hip stem defines an overall stem axis extending distally and alignable with the anatomical axis A after implantation, and further includes a distal end having a local stem axis that is parallel to, but medially offset from, the overall stem axis. A distal centralizer is securable to the distal end with an interference fit and includes a first surface configured to engage the anatomical canal and define a first proximal-distal axis, and a second surface configured to engage the distal end of the hip stem and define a second proximal-distal axis. The first axis is laterally offset from the second axis by an amount corresponding to the medial offset between the local stem axis and the overall stem axis, such that, when the distal centralizer is attached and the hip stem is inserted into the anatomical canal, the distal end of the hip stem is centralized to align the overall stem axis with the anatomical axis.

According to certain implementations, the distal centralizer includes a proximal component and a distal component. The distal component includes the first surface and defines a concave profile having a centerpoint that is alignable with the overall stem axis when the distal centralizer is attached to the hip stem. The concave profile extends arcuately and symmetrically from the centerpoint to define increasing diameters at proximally spaced locations along the distal component, with at least one of the diameters configured to engage opposing portions of the anatomical canal. The proximal component includes a receptacle having a proximally oriented opening sized to receive the distal end of the hip stem in an interference fit. The receptacle includes an inner wall having a bottom point that is medially offset from the centerpoint by an amount corresponding to the medial offset between the local stem axis and the overall stem axis, and the inner wall is configured to mate with opposing surfaces of the distal end of the hip stem, such that the inner wall defines the second surface of the distal centralizer.

In still further implementations, the hip stem includes a cylindrical post secured to the distal end of the hip stem and extending distally therefrom. The cylindrical post includes an outer cylindrical wall and a circumferential cross-section characterized by a central post axis extending longitudinally through the center of the cross-section. The post includes a base secured to the distal end of the hip stem, the base having a central base axis that extends distally and is laterally offset from the local stem axis by an amount corresponding to the medial offset between the local stem axis and the overall stem axis. The distal centralizer further includes a bushing having an inner wall that defines a bore sized to receive the cylindrical post in an interference fit. The bushing includes a resiliently compressible outer concave surface oriented distally and having a profile that extends symmetrically from the central base axis to define increasing diameters in the proximal direction. The base includes the first surface of the distal centralizer, and the outer concave surface of the bushing defines the second surface, such that the interaction between the post and the bushing aligns the central base axis with the anatomical axis.

In further possible implementations, the distal centralizer includes a plug having inner and outer plug walls. The inner plug wall defines a plug aperture having a plug aperture axis that is alignable with the local stem axis when the plug is received on the hip stem. The inner plug wall corresponds to the first surface of the distal centralizer, and the plug aperture axis corresponds to the first proximal-distal axis and the local stem axis, such that the plug aperture axis is offset medially from the overall stem axis when the plug is received on the distal end. The second surface of the distal centralizer is defined by the outer plug wall and is configured to engage the anatomical canal. The outer plug wall is spaced asymmetrically relative to the inner plug wall and defines a central outer wall axis that is offset from the plug aperture axis by an amount corresponding to the medial offset between the local stem axis and the overall stem axis. In certain implementations, the inner plug wall extends proximally from the plug to increase engagement with the distal end of the hip stem.

In yet another potential implementation, the distal end of the hip stem includes portions that define a bore extending longitudinally and terminating in a receiving aperture, the bore having a central bore axis aligned with the overall stem axis. The distal centralizer includes a proximal component having a shank with a central shank axis, the shank extending proximally from a distal component and being sized to be received through the receiving aperture and into the bore. The shank axis is medially offset from the central bore axis by an amount corresponding to the medial offset between the local stem axis and the overall stem axis, such that engagement between the shank and the bore, together with the distal component engaging the anatomical canal, enables alignment of the hip stem with the anatomical axis upon insertion.

### BRIEF DESCRIPTION OF THE DRAWINGS

The accompanying drawings, which are incorporated herein and constitute part of this specification, illustrate the presently preferred embodiments of the disclosure, and, together with the general description given above and the detailed description given below, serve to explain the features of the disclosure. In the drawings:

FIG. 1A shows a side view of a tri-taper hip stem, according to some embodiments;

FIG. 1B shows a front view of a tri-taper hip stem, according to some embodiments;

FIG. 1C shows a top view of a tri-taper hip stem, according to some embodiments;

FIG. 2 shows an outline overlay of a cemented tri-taper hip stem and a cementless tri-taper hip stem, according to some embodiments; and

FIG. 3 shows a side view of a tri-taper hip stem defining various dimensions, according to some embodiments.

FIG. 4 is a side elevational view of one possible implementation of components of a hip implant system according to the present disclosure;

FIG. 5 is a cross-sectional view of the hip implant system of FIG. 4;

FIG. 6 is an enlarged, isometric view of one possible implementation of a distal centralizer of the hip implant system of FIGS. 4-5;

FIG. 7 is a cross-sectional view of the distal centralizer of FIG. 6;

FIG. 7 is a side elevational view of the hip stem of the hip implant system;

FIG. 8A is a top plan view of the implementation of a distal centralizer shown in FIG. 6;

FIG. 8B is a cross-sectional view of the distal end of the hip stem taken along line A-A of FIG. 7B;

FIG. 9A is a side elevational view of a distal portion of a hip stem;

FIG. 9B is a cross-sectional view of a distal centralizer corresponding to the hip stem of FIG. 9A;

FIG. 10 is a side elevational view of a hip stem including a distal post according to another implementation;

FIG. 11 is an isometric view of a distal centralizer configured to engage the distal post of FIG. 10;

FIG. 12 is a front elevational view of the distal post of FIG. 10;

FIG. 13 is a top plan view of a distal centralizer according to another implementation;

FIG. 14 is a side elevational view of a distal centralizer;

FIG. 15 is a cross-sectional view of a hip stem and distal centralizer according to another implementation;

FIG. 16A is an isometric view of a plug-type distal centralizer;

FIG. 16B is a top plan view of the distal centralizer of FIG. 16A;

FIG. 16C is a cross-sectional view of the distal centralizer of FIG. 16A;

FIG. 17A is an isometric view of another implementation of a plug-type distal centralizer;

FIG. 17B is a top plan view of the distal centralizer of FIG. 17A;

FIG. 17C is a cross-sectional view of the distal centralizer of FIG. 17A;

FIG. 18 is an isometric view of another implementation of a distal centralizer, wherein the inner plug wall extends proximally from the plug;

FIG. 19 is a cross-sectional view of a distal centralizer engaged with a distal portion of a hip stem;

FIG. 20 is a cross-sectional view of a hip stem including a distal centralizer according to another implementation; and

FIG. 21 is an isometric view of a distal centralizer according to another implementation.

### DETAILED DESCRIPTION

Provided herein are both cementless and cemented tri-taper hips stems for use in a hip arthroplasty. The hip stems described herein are designed to minimize the chances of subsidence which can occur when the bone near an implanted device settles or collapses and can cause a periprosthetic fracture, patient pain, joint dislocation, and need for revision surgery. In the case of subsidence of a hip stem, subsidence can occur when the fit of the hip stem is not tight enough, particularly in the case of a press fit or cementless stem.

Accordingly, the hip stems described herein have a taper design and, in some embodiments, a medial collar, both of which are designed to help reduce instances of subsidence. Each of these features are designed in more detail below.

First, the tri-taper hip stems described herein help prevent subsidence with a tri-taper design by filling the patient's femoral canal along more surfaces than other hip stems. Filling the femoral canal by providing more surface area contact between the hip stem and the femoral canal can provide a tighter fit, or a tighter press fit, of the hip stem into the patient's femur. The tightness of the fit of the hip stem in the patient's femoral canal is directly related to the chances of subsidence. Therefore, a tighter fight formed by providing more surface area contact between the surfaces of the hip stem and the bone of the femoral canal will decrease the likelihood of subsidence.

The higher surface area contact between the surfaces of the hip stem and the femoral canal is due to the taper design of the hip stem (“tri-taper”). These three different tapers can provide a tighter press fit due to the contact between the hip stem and the cortical bone in not only the medial and lateral aspect, which is typical in other hip stems, but also in the anterior and posterior aspects. The additional surface area contact particularly in the anterior and posterior aspects can help form a tighter fit or a tighter press fit of the hip stem in the femoral canal, minimizing the risk of subsidence.

Second, the tri-taper hip stems described herein can also help prevent subsidence by transferring the forces driving subsidence to the calcar of the femur, allowing the hip stem to stay in place and reduce the amount of subsidence. To transfer these forces, some embodiments of the hip stems described herein may include a medial collar. This medial collar is located at a proximal end of the hip stem between the body of the hip stem and a neck of the hip stem. The medial collar is optional.

During a total hip arthroplasty (THA), a femoral neck osteotomy may be performed to remove the femoral head after the correct size stem has been estimated, for example, in pre-operative templating. Following this, a reamer may be used to locate the femoral canal and broaching occurs until the cortical walls of the femur have been contacted. The broach sizes correlate to the size stems available. The surgeon utilizes the appropriate broach to choose the stem size more accurately. After a trial reduction is conducted to ensure the correct femoral head and neck offset is chosen, the stem is inserted into the broached canal. Trial reduction occurs with the broach left in the metaphysis. This may be used to assess leg length, range of motion, and joint stability. A cementless implant, after impaction, creates a press fit against the implant and the cortical walls of the femur. The stems have numerous sizes which may vary in the medial/lateral, anterior/posterior, and/or proximal/distal aspects. These size variations allow for a wide range of patients to receive the size stem most appropriate to their anatomy.

More recently, surgeons have been conducting total hip arthroplasties using a direct anterior approach. This approach can introduce a difficult angle for surgeons to insert the hip stem into the medullary canal. In some embodiments, a tri-taper hip stem according to some embodiments described herein may be designed with a relatively shorter stem with material relief on the lateral side of the distal tip. This geometry may allow the stem to be more easily inserted at the angle necessary when using a direct anterior approach.

Another benefit of the tri-taper hip stems described herein is that both the cementless tri-taper hip stems and the cemented tri-taper hip stems utilize the same broach system. This reduces the amount of instrumentation needed for the procedure and streamlines the entire process. It can also save operating room time, and in some cases, even preserves a patient's bone. In some embodiments, the cemented and cementless hip stems described herein may also utilize the same inserter(s), trial(s), and/or femoral head(s), further improving efficiency and decreasing the amount of instrumentation associated with the implant(s).

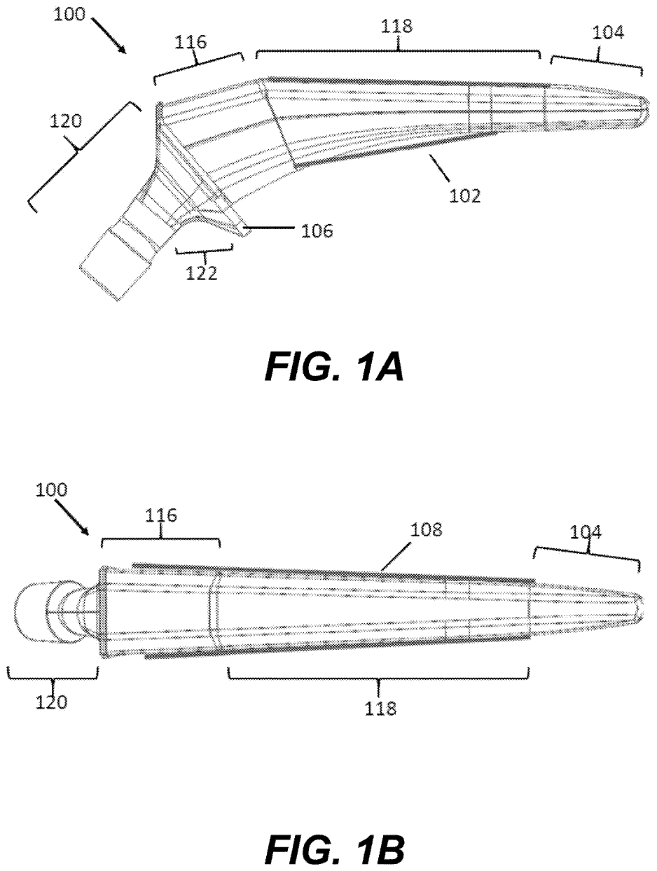

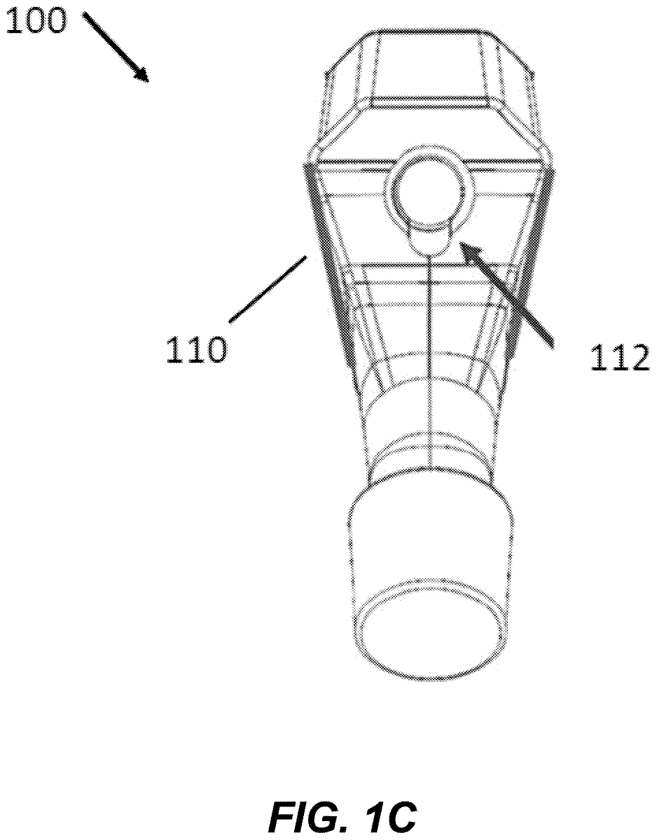

FIGS. 1A-1C show a side view, a front view, and a top view of a tri-taper hip stem 100 according to some embodiments. Each view is described in detail below.

FIG. 1A shows a side view of a tri-taper hip stem 100, according to some embodiments. Shown in this figure is a first taper 102, a distal end 104, and a medial collar 106. Also shown are proximal portion 116, middle portion 118, neck portion 120, and base 122 of neck portion 120.

As shown, hip stem 100 extends from neck portion 120 to distal end 104. Base 122 of neck portion 120 is located on a proximal portion of the neck portion 120. The proximal portion 116, middle portion 118, and distal portion 104 form the stem body. The proximal portion 116 is located immediately distal to the base 122 of neck portion 120 or medial collar 106, depending on whether the medial collar 106 is included. The middle portion 118 is immediately distal to proximal portion 116, and distal end 104 (or distal portion 104) is immediately distal to middle section 118.

In some embodiments, the length of middle portion 118 is greater than a length of each of proximal portion 116 and distal portion 104. In some embodiments, the length of middle portion 118 is at least double the length of proximal portion 116. In some embodiments, the length of middle portion 118 is at least double the length of distal portion 104. As used herein, the “length” of an individual portion of the stem body (e.g., proximal portion 116, middle portion 118, or distal portion 104) is measured along an axis extending through the center of the portion, generally (but not necessarily strictly) from the proximal end to the distal end (or in the cranial-caudal direction). By this definition, the axis of each of the proximal portion 116, the middle portion 118, and the distal portion 104 as shown in FIG. 1A runs along a different plane and no two axes of the three axes are co-axial.

In some embodiments, the lateral surface or edge of the proximal portion 116 runs along a first plane and the lateral surface or edge of middle portion 118 runs along a second plane, wherein the first plane and the second plane are not parallel and are not perpendicular. In some embodiments, the lateral surface or edge of the proximal portion 116 runs along a first plane and the lateral surface or edge of the distal portion 104 runs along a third plane, wherein the first plane and the third plane are not parallel and are not perpendicular. In some embodiments, the second plane and the third plane are not parallel and are not perpendicular. In some embodiments, the medial edge or surface of the proximal portion 116 runs along a fourth plane and the medial edge or surface of the middle portion 118 runs along a fifth plane, wherein the fourth plane and the fifth plane are not parallel and are not perpendicular. In some embodiments, the medial edge or surface of the proximal portion 116 runs along the fourth plane and the medial edge or surface of the distal portion 104 runs along a sixth plane, wherein the fourth plane is not parallel and is not perpendicular to the sixth plane. In some embodiments, the fifth plane and the sixth plane are not parallel and are not perpendicular. In some embodiments, no two planes of the six total planes (the first plane, the second plane, the third plane, the fourth plane, the fifth plane, and the sixth plane) are parallel. In some embodiments, no two planes of the six total planes (the first plane, the second plane, the third plane, the fourth plane, the fifth plane, and the sixth plane) are perpendicular.

The tri-taper design is described below with respect to the first taper 102, the second taper 108, and the third taper 110. Each taper “extends” insofar as each of the involved surfaces of the hip stem 100 extend planarly. (However, by definition, the first surface of a taper will extend along a different plane than the second, opposing surface of said taper.) In one example, the first taper 102 as shown in FIG. 1A extends only along the middle portion 118. As is shown in the figure, and particularly on the lateral side of the hip stem 100, the lateral surface of proximal portion 116 and the lateral surface of distal portion 104 extend along different planes than the plane of the first taper 102 of the lateral surface of middle portion 118 of hip stem 100. However, as described in further detail below, this is only one embodiment of hip stem 100 and first taper 102.

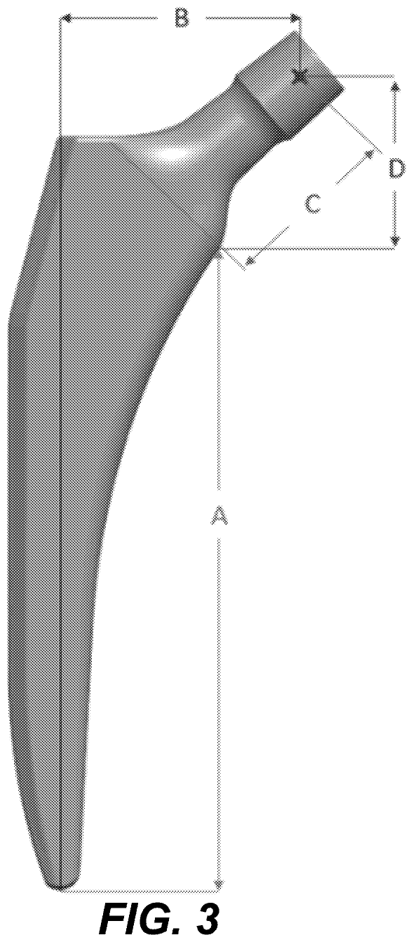

The first taper 102 extends along a length of the hip stem 100 such that a width of the hip stem in the medial-lateral direction narrows as the first taper 102 extends towards the distal portion of the hip stem 100. As used herein the “length of the hip stem” is measured in the direction labeled “A” in FIG. 3. (Note that this is different than a “length” of an individual portion of the stem body.) In some embodiments, the first taper 102 extends through the proximal portion of the hip stem 100. In some embodiments, the first taper extends only through the proximal portion of the hip stem 100. In some embodiments, the taper angle of the first taper 102 is the same along both the medial edge and lateral edge of the hip stem 100. In some embodiments, the taper angle of the first taper 102 is different along each of the medial edge and the lateral edge of the hip stem 100. In some embodiments, the taper angle of the first taper 102 along the medial edge of the hip stem 100 is greater than the taper angle of the first taper 102 along the lateral edge of the hip stem 100. In some embodiments, the medial edge of the hip stem 100 is curved.

In some embodiments, the first taper 102 reduces the width of the hip stem in the medial-lateral direction by 20-60% or by 30-50%. In some embodiments, the first taper 102 reduces the width of the hip stem in the medial-lateral direction by less than or equal to 60%, less than or equal to 55%, less than or equal to 50%, less than or equal to 45%, less than or equal to 40%, less than or equal to 35%, less than or equal to 30%, or less than or equal to 25%. In some embodiments, the first taper 102 reduces the width of the hip stem in the medial-lateral direction by greater than or equal to 20%, greater than or equal to 25%, greater than or equal to 30%, greater than or equal to 35%, greater than or equal to 40%, greater than or equal to 45%, greater than or equal to 50%, or greater than or equal to 55%.

The distal end 104 of the hip stem features a taper in the medial-lateral aspect to relieve material for easy insertion of the stem. This relief cut may be featured on the lateral side of the hip stem 100. As explained above, a direct anterior approach, which is being use more frequently, introduces a challenging angle for hip stem insertion due to patient anatomy. However, a taper in the medial-lateral aspect on the distal end 104 of the hip stem 100 can help a surgeon more easily insert the hip stem into the medullary canal of a patient's femur.

Some embodiments of a tri-taper hip stem 100 can include a medial collar 106. The medial collar 106 is optional, and it is designed to distribute axial forces over the calcar plane in order to minimize the possibility of implant subsidence. More specifically, the medial collar 106 is designed to sit on the calcar of the patient's femur when properly implanted. If the hip stem begins to subside, forces will be transferred to the calcar and the hip stem will stay in place.

FIG. 1B shows a front view of a tri-taper hip stem 100, according to some embodiments. FIG. 1B specifically shows the second taper 108 of the tri-taper design, as well as the neck portion 120, proximal portion 116, middle portion 118, and distal portion 104.

The second taper 108 extends along the length of the hip stem 100 such that a width of the hip stem in the posterior-anterior direction narrows as the second taper 108 extends towards the distal portion of the hip stem 100. As used herein the “length of the hip stem” is measured in the direction labeled “A” in FIG. 3. (Note that this is different than a “length” of an individual portion of the stem body.) In some embodiments, the second taper 108 extends along the proximal portion, the middle portion, and the distal portion of the hip stem 100. In some embodiments, the second taper 108 extends only along the proximal portion and the middle portion of the hip stem 100. In some embodiments, the taper angle along the anterior surface of the hip stem 100 is the same as the taper angle along the posterior surface of the hip stem 100. In some embodiments, the taper angle along the anterior surface of the hip stem 100 is different than the taper angle along the posterior surface of the hip stem 100.

In some embodiments, the second taper 108 reduces the width of the hip stem 100 in the posterior-anterior direction by 20-50%. In some embodiments, the second taper 108 reduces the width of the hip stem 100 in the posterior-anterior direction by less than or equal to 50%, less than or equal to 45%, less than or equal to 40%, less than or equal to 35%, less than or equal to 30%, or less than or equal to 25%. In some embodiments, the second taper 108 reduces the width of the hip stem 100 in the posterior-anterior direction by greater than or equal to 20%, greater than or equal to 25%, greater than or equal to 30%, greater than or equal to 35%, greater than or equal to 40%, or greater than or equal to 45%.

FIG. 1C shows a top view of a tri-taper hip stem 100, according to some embodiments. Specifically, FIG. 1C shows the third taper 110 and a key slot inserter hole 112.

The third taper 110 of the tri-taper design is designed to engage the press fit in the anterior and posterior cortical bone. Many hip stems are only configured to press fit in the medial and lateral directions. However, the third taper allows the tri-taper hip stem 100 to achieve a press fit in not only the medial and lateral aspects, but also in the anterior and posterior aspects. Achieving a press fit in the medial and lateral direction as well as in the anterior and posterior directions allow for a tighter fit of the hip stem and a reduced chance of subsidence.

In some embodiments, the third taper 110 of hip stem 100 extends along an axis of the proximal portion of hip stem 100 such that a width of the hip stem in the medial-lateral direction widens as the third taper 110 extends from the stem neck 120 towards the middle portion 118 of the hip stem 100. In some embodiments, the third taper 110 extends through the proximal portion of the hip stem 100. In some embodiments, the third taper 110 extends only through the proximal portion 116 of the hip stem 100. In some embodiments, the taper angle of the third taper 110 is the same along both the posterior surface and the anterior surface of the proximal portion 116 of hip stem 100. In some embodiments, the taper angle of the third taper 110 is the same along each of the posterior surface and the anterior surface of the hip stem 100. In some embodiments, the taper angle of the third taper 110 along the posterior surface of the hip stem 100 is different than the taper angle of the third taper 110 along the anterior surface of the hip stem 100.

In some embodiments, the third taper 110 enlarges the width of the hip stem 100 in the posterior-anterior direction by 20-80% or 10-40%. In some embodiments, the third taper 110 enlarges the width of the hip stem 100 in the posterior-anterior direction by less than or equal to 80%, less than or equal to 70%, less than or equal to 60%, less than or equal to 50%, less than or equal to 40%, less than or equal to 30%, or less than or equal to 20%. In some embodiments, the third taper 110 enlarges the width of the hip stem 100 in the posterior-anterior direction by greater than or equal to 10%, greater than or equal to 20%, greater than or equal to 30%, greater than or equal to 40%, greater than or equal to 50%, greater than or equal to 60%, or greater than or equal to 70%.

In some embodiments, a tri-taper hip stem 100 may include a key slot inserter hole 110. The key slot inserter hole 112 can allow for version control of the implant during insertion. Version control refers to the position of the hip stem 100 in the femoral canal. If the hip stem 100 is not placed correctly, it will be anteverted or retroverted. The key slot inserter hole 112 can help a surgeon maintain proper alignment during insertion and reduce the chances of an anteverted or retroverted implant. In some embodiments, a key slot can be machined in the medial-lateral aspect in addition to the proximal threaded drive pin hole.

Further, some embodiments of the hip stems described herein may include one or more surface coatings or surface treatments. Surface coatings can include, for example, a titanium plasma spray and/or a hydroxyapatite coating. In some embodiments, a portion of a hip stem might be coated with a titanium plasma spray coating. The portion of the hip stem coated with a titanium plasma spray may comprise the stem body. More specifically, it may comprise a proximal portion (e.g., proximal portion 116) of the stem body of the hip stem. In some embodiments, the titanium plasma spray may also coat a portion of or the entire surface of the middle portion (e.g., middle portion 118) of the hip stem. In some embodiments, a hip stem may be entirely coated with a hydroxyapatite coating or only partially coated with a hydroxyapatite coating. In some embodiments, a stem body of a hip stem may be entirely coated with a hydroxyapatite coating. In some embodiments, a stem neck (including a base of the stem neck) may not be coated with a hydroxyapatite coating. In some embodiments, only cementless hip stems may include one or more surface coatings. In some embodiments, both cementless and cemented hip stems may include one or more coatings. The titanium plasma spray coating can improve fixation (both short-term and long-term fixation). The hydroxyapatite coating can promote bone regrowth. In some embodiments, a tri-taper hip stem may comprise both a titanium plasma spray coating and an HA coating. In some embodiments, a tri-taper hip stem may comprise only one of a titanium plasma spray coating or an HA coating.

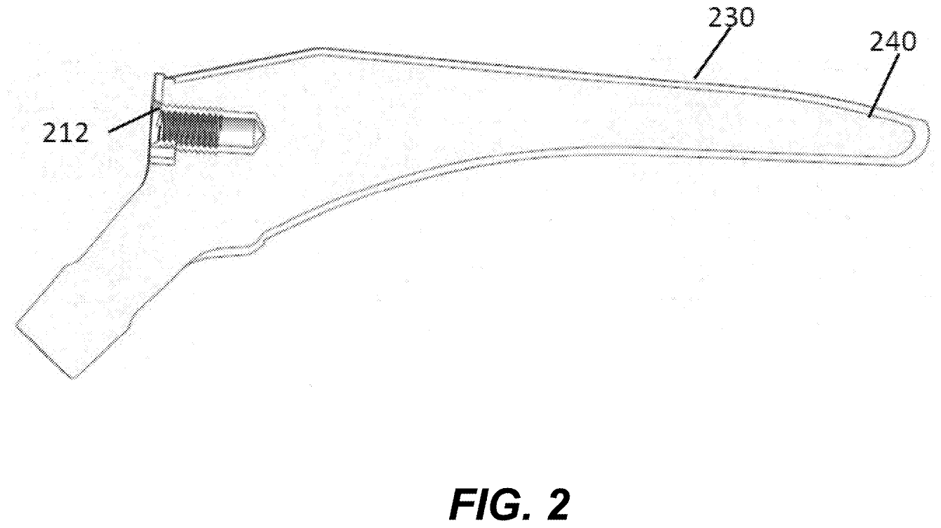

FIG. 2 shows an overlay of a cementless tri-taper hip stem 230 and a cemented tri-taper hip stem 240 of the same size. As used herein, “same size” means that a cementless hip stem 230 that is the “same size” as a cemented hip stem 240 may be used interchangeably in a patient with a specific anatomy. However, as shown in FIG. 2, the cemented hip stem 240 is slightly smaller than the cementless hip stem 230 to allow space for the cement to fit between the patient's bone and the implant. In some embodiments, the cemented hip stem 240 may be 2 mm smaller around all surfaces of the stem body to allow for the cement mantle. However, the neck 120 and base 122 may be the same dimensions between the cementless hip stem 230 and the cemented hip stem 240 such that all they are both compatible with existing femoral heads (e.g., Globus Medical's existing femoral heads). Having the cemented option that uses the same broaches as the press fit stem will reduce the amount of instrumentation that hospitals and surgery centers need to have. The hip stems described herein (both cementless and cemented) may also utilize the same inserter(s), trial neck(s), and/or femoral head(s), further reducing the necessary amount of instrumentation. It will also save surgeons time in the operating room when they decide to switch to a cemented stem since they won't have to re-broach or ream and will preserve the patient's bone.

The hip stems described herein may comprise one or more biocompatible materials. For example, a hip stem according to some embodiments may be made from a metal (e.g., titanium, stainless steel, cobalt chrome, carbon composite, or suitable alloys), a plastic or polymer (e.g., polyethylene, ultra-high molecular weight polyethylene (UHMWPE), polyetheretherketone (PEEK)), or combinations of such materials. These parts may be machined, constructed from additive manufacturing, such as 3D printing, subtractive manufacturing, or hybrid manufacturing processes. Although the materials described herein are exemplified, it will be appreciated that any suitable materials and construction may be selected for the individual components.

### Kits

Also provided herein are kits for use in a hip arthroplasty. In some embodiments, a kit can include a broaching system and two or more hip stems. The broaching system may be configured for preparing a femur for a total hip arthroplasty. The two or more hip stems may include one or more of: two or more sizes of cementless hip stems, two or more sizes of cemented hip stems, or a cementless hip stem and a cemented hip stem of the same size. In some embodiments, the kit comprises all of: two or more sizes of cementless hip stems, two or more sizes of cemented hip stems, and a cementless hip stem and a cemented hip stem of the same size. In some embodiments, all of the hip stems in the kit comprise a stem body having a length formed by a proximal portion, a middle portion, and a distal portion; and a stem neck extending from the proximal portion of the stem body and terminating at a free end, wherein the stem portion comprises a first taper along the length of the stem body causing a width of the stem body in the medial-lateral direction to decrease as the first taper extends along the middle portion of the stem body from a proximal end of the middle portion to a distal end of the middle portion, a second taper along the length of the stem body causing a width of the stem body in the posterior-anterior direction to decrease as the second taper extends along the middle portion of the stem body from the proximal end of the middle portion to the distal end of the middle portion, and a third taper along a base of the stem neck causing a width of the base in the posterior-anterior direction to increase as the third taper extends along the base from a proximal end of the stem neck towards the stem body, and wherein the broaching system can be used for both the cemented hip stem and the cementless hip stem.

The kits provided herein can also include other implants (e.g., acetabular shell, liner, femoral head), various instruments, and other components for performing the procedure.

### Methods

Provided herein are methods for using the hip stems described in detail above. In some embodiments, a method for a hip arthroplasty may include one or more of the following steps in any suitable order: (1) performing a femoral neck osteotomy to remove a femoral head of a femur; (2) inserting a reamer into a femoral canal and broaching the bone until cortical walls of the femur are contacted; (3) trialing a reduction to assess leg length and/or joint stability; and (4) impacting a cementless tri-taper hip stem into the femoral canal.

In some embodiments, a method for a hip arthroplasty may include one or more of the following steps in any suitable order: (1) performing a femoral neck osteotomy to remove a femoral head of a femur; (2) inserting a reamer into a femoral canal and broaching the bone until cortical walls of the femur are contacted; (3) trialing a reduction to assess leg length and/or joint stability; (4) filling the femoral canal with cement; and (5) inserting a cemented tri-taper hip stem into the femoral canal.

### EXAMPLES

### Example 1: Press Fit (Cementless) Tri-Taper Hip Stems

Table 1, below, shows specific dimensions for cemented tri-taper hip stems sizes 1-12. The dimensions A, B, C, and D refer to the measurements labeled in FIG. 3.

TABLE 1
Cemented Tri-Taper Hip Stems Sizes 1-12
Press Fit Stems

| | | B | | | | | D |
|---|---|---|---|---|---|---|---|
| | | Horizontal offset from | | | C | | Vertical |
| | A | Stem Centerline (Offset) | | | Neck Length | | Offset |
| Size | Length | STD | LAT | Offset | STD | LAT | (Leg Length) |
| 1 | 97 | 36.2 | 42.2 | 6 | 28 | 32 | 27 |
| 2 | 99 | 36.2 | 42.2 | 6 | 28 | 32 | 27 |
| 3 | 101 | 37.4 | 43.4 | 6 | 29 | 33.5 | 28 |
| 4 | 103 | 37.4 | 43.4 | 6 | 29 | 33.5 | 28 |
| 5 | 105 | 38.6 | 44.6 | 6 | 30.5 | 34.75 | 29 |
| 6 | 107 | 38.6 | 44.6 | 6 | 30.5 | 34.75 | 29 |
| 7 | 109 | 39.75 | 45.75 | 6 | 32 | 36 | 30 |
| 8 | 111 | 39.75 | 45.75 | 6 | 32 | 36 | 30 |
| 9 | 113 | 40.9 | 46.9 | 6 | 33 | 37.5 | 31 |
| 10 | 115 | 40.9 | 46.9 | 6 | 33 | 37.5 | 31 |
| 11 | 117 | 42.1 | 48.1 | 6 | 34.5 | 38.8 | 32 |
| 12 | 119 | 42.1 | 48.1 | 6 | 34.5 | 38.8 | 32 |

Table 2 below shows specific dimensions for cementless tri-taper hip stems sizes 1-12. The dimensions A, B, C, and D refer to the measurements labeled in FIG. 3. As seen in the table, the actual dimensions of a cemented hip stem of a given size are smaller than the corresponding cementless (press fit) hip stem of the same size.

TABLE 2
Cementless Tri-Taper Hip Stems Sizes 1-12
Cemented Stems

| | | B | | | | | D |
|---|---|---|---|---|---|---|---|
| | | Horizontal offset from | | | C | | Vertical |
| | A | Stem Centerline (Offset) | | | Neck Length | | Offset |
| Size | Length | STD | LAT | Offset | STD | LAT | (Leg Length) |
| 3 | 95 | 37.4 | 43.4 | 6 | 29 | 33.5 | 28 |
| 4 | 97 | 37.4 | 43.4 | 6 | 29 | 33.5 | 28 |
| 5 | 101 | 38.6 | 44.6 | 6 | 30.5 | 34.75 | 29 |
| 6 | 103 | 38.6 | 44.6 | 6 | 30.5 | 34.75 | 29 |
| 7 | 105 | 39.75 | 45.75 | 6 | 32 | 36 | 30 |
| 8 | 107 | 39.75 | 45.75 | 6 | 32 | 36 | 30 |
| 9 | 109 | 40.9 | 46.9 | 6 | 33 | 37.5 | 31 |
| 10 | 111 | 40.9 | 46.9 | 6 | 33 | 37.5 | 31 |
| 11 | 113 | 42.1 | 48.1 | 6 | 34.5 | 38.8 | 32 |
| 12 | 115 | 42.1 | 48.1 | 6 | 34.5 | 38.8 | 32 |

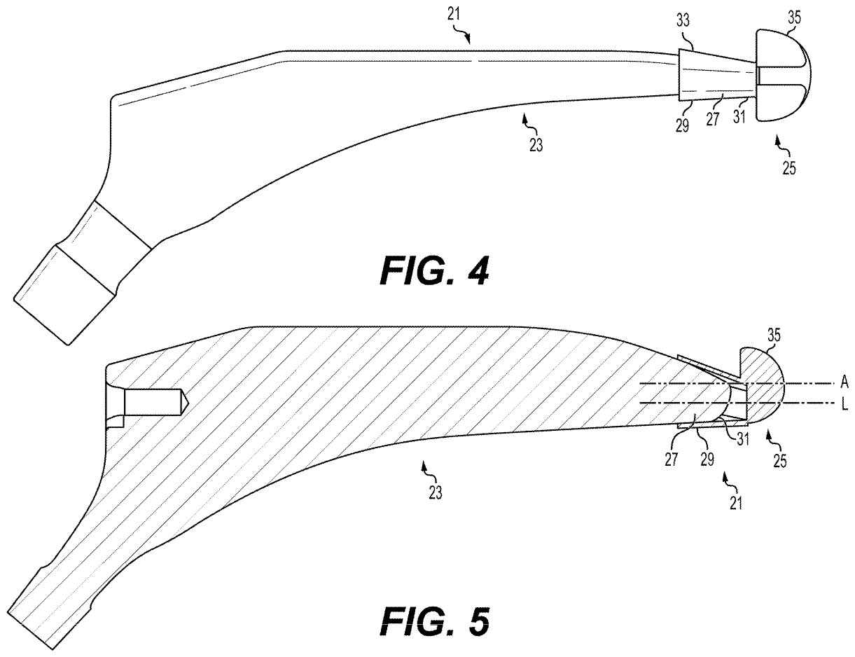

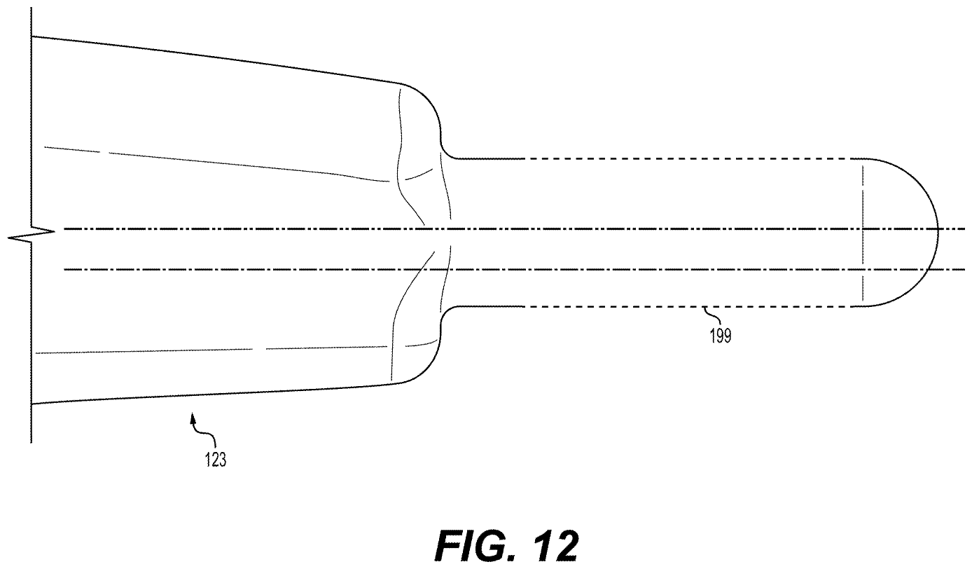

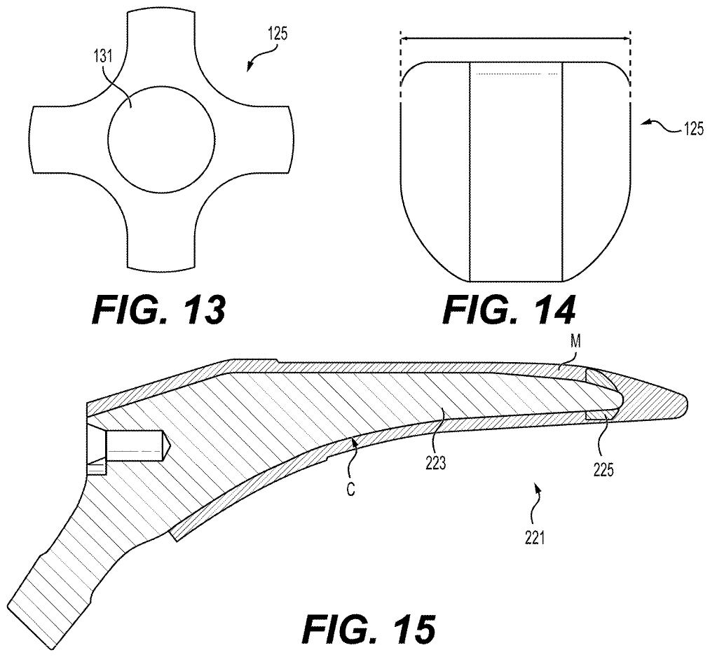

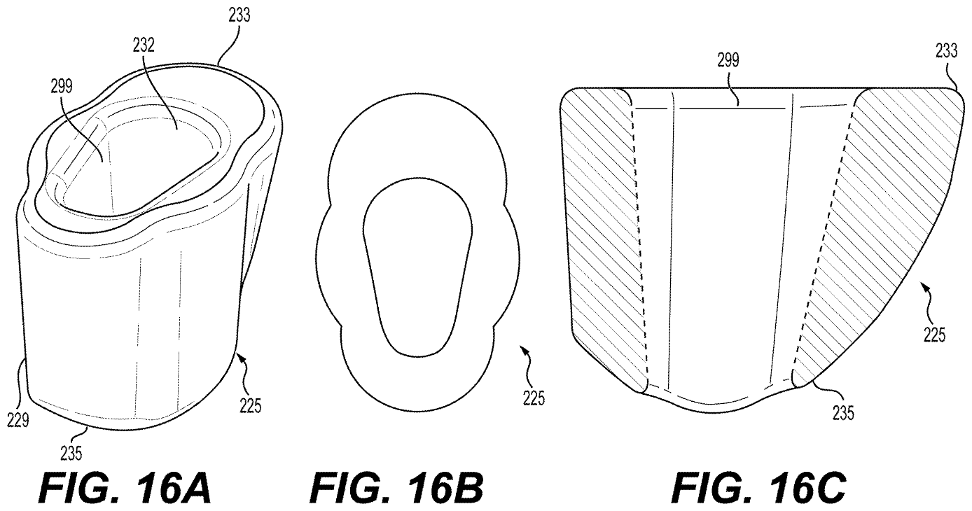

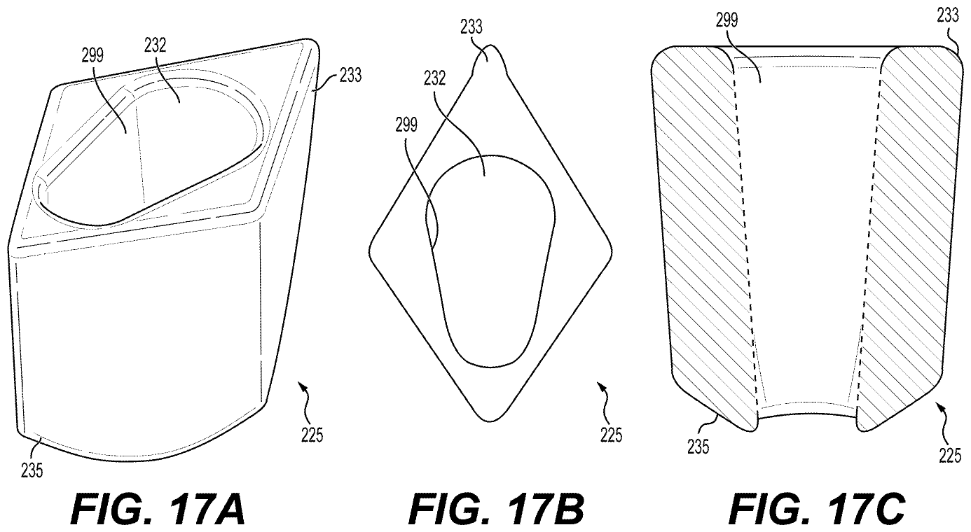

FIGS. 4-21 illustrate various further implementations of a hip implant system for use with any of a variety of arthroplasty procedures. Referring to FIGS. 4 to 9A, 9B, a hip implant system 21 includes a hip stem 23 and a distal centralizer 25 removably attachable to the distal end 27 of hip stem 23. The hip stem 23 extends distally from a proximal base portion and is configured to be inserted into and secured within a femoral canal of a patient. The hip stem 23 defines an overall stem axis extending along a proximal-distal direction and alignable with an anatomical axis A of the femur following implantation. The distal end 27 of the hip stem defines a local stem axis L that is generally parallel to the overall stem axis and is offset medially relative thereto.

The distal centralizer 25 includes a first surface 29 configured to engage the anatomical canal and a second surface 31 configured to engage the distal end 27 of the hip stem 23. The first surface 29 defines a first proximal-distal axis, while the second surface 31 defines a second proximal-distal axis. The first and second axes are laterally offset relative to one another by an amount corresponding to the medial offset between the local stem axis L and the overall stem axis of the hip stem 23, such that, upon assembly and insertion, the distal centralizer 25 positions the distal end 27 to align the overall stem axis with the anatomical axis A of the femur.

In the implementation shown in FIGS. 4-9A, 9B, the distal centralizer 25 includes a proximal component 33 and a distal component 35. The distal component 35 includes the first surface 29, which is defined by a concave outer profile having a centerpoint 37 that is alignable with the overall stem axis when the distal centralizer 25 is attached to the hip stem 23. The concave profile extends arcuately and symmetrically from the centerpoint 37 to define increasing diameters in a proximal direction, such that portions of the distal component 35 engage opposing regions of the anatomical canal to centralize the hip stem 23. In certain implementations, the distal component 35 includes a generally hemispherical outer geometry to facilitate insertion and uniform engagement with the canal.

The proximal component 33 includes a receptacle 39 having a proximally oriented opening configured to receive the distal end 27 of the hip stem 23 in an interference fit. The receptacle 39 includes an inner wall 41 that defines the second surface 31 and is configured to mate with corresponding outer surfaces of the distal end 27. The inner wall 41 includes geometries corresponding to medial/lateral taper surfaces, anterior/posterior taper surfaces, and proximal/distal taper features of the distal end 27, thereby providing a close conforming engagement between the distal centralizer 25 and the hip stem 23. The inner wall 41 further includes a bottom point that is medially offset relative to the centerpoint 37 of the distal component 35, such that the offset between the first and second surfaces 29, 31 corresponds to the offset between the local stem axis L and the overall stem axis.

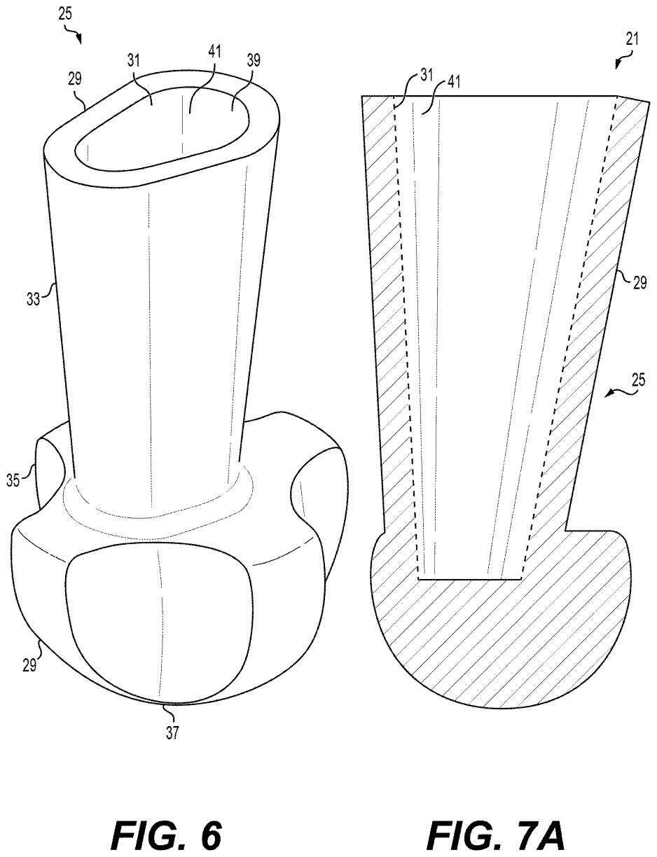

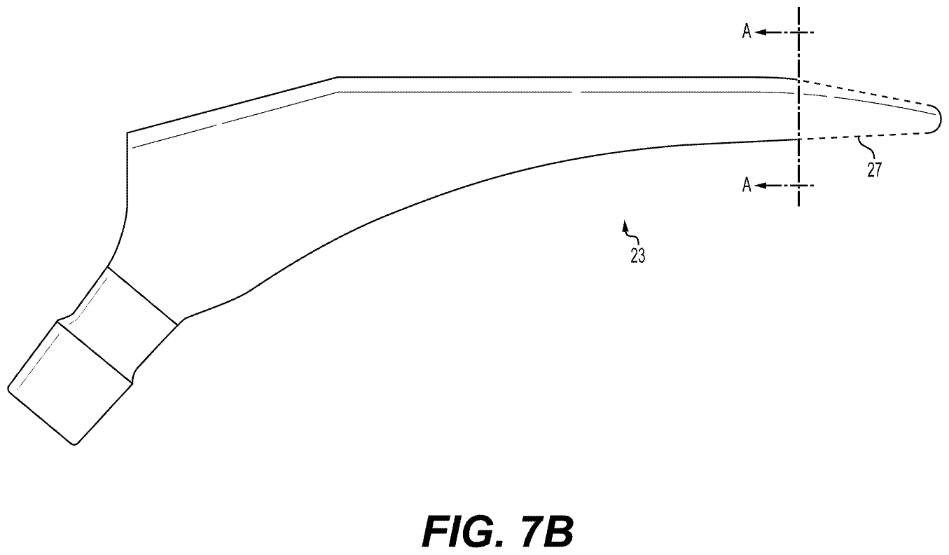

As shown in FIG. 6 and FIG. 7A, the receptacle 39 is shaped to receive the tri-taper geometry of the distal end 27, enabling stable attachment such that the distal centralizer 25 can support its own weight when assembled with the hip stem 23. The interference fit between the receptacle 39 and the distal end 27 allows for secure positioning during insertion into the femoral canal while still permitting removal if desired.

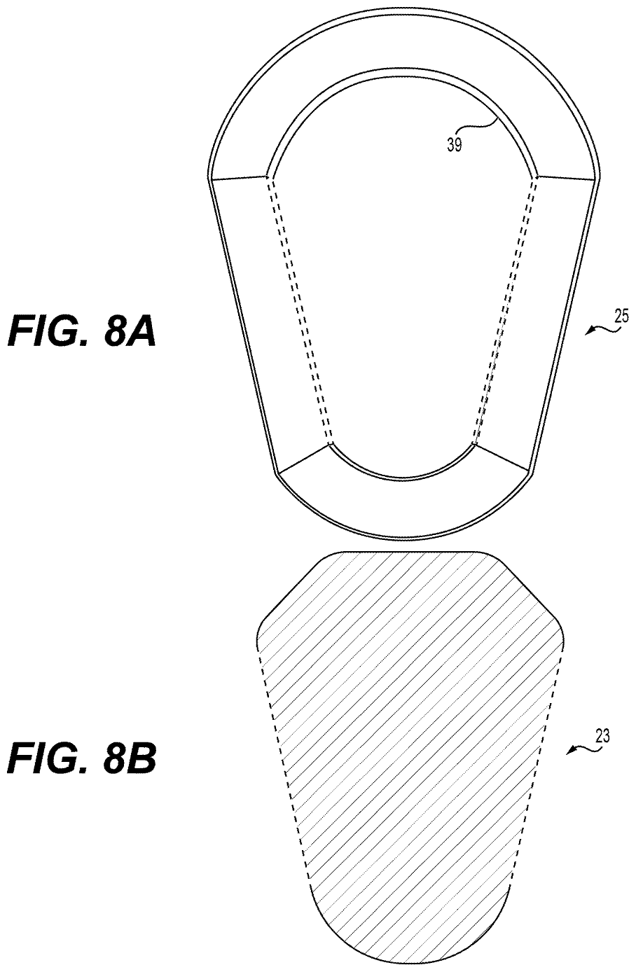

Referring to FIGS. 8A and 8B, the distal centralizer 25 (FIG. 8A) may be provided in a range of sizes corresponding to different sizes of hip stems 23, wherein the surfaces shown in FIG. 8A may create an internal geometry of the receptacle 39 for distal component 35 which varies to match corresponding dimensions and contours of medial/lateral, anterior/posterior, and proximal/distal aspects of the corresponding the size of hip stem 23 as seen in FIG. 8B. In certain implementations, the distal centralizer 25 is sized such that the distal component 35 fits within a cavity formed by a broach used to prepare the femoral canal, thereby enabling the formation of a substantially uniform cement mantle around the distal end 27 of the hip stem 23.

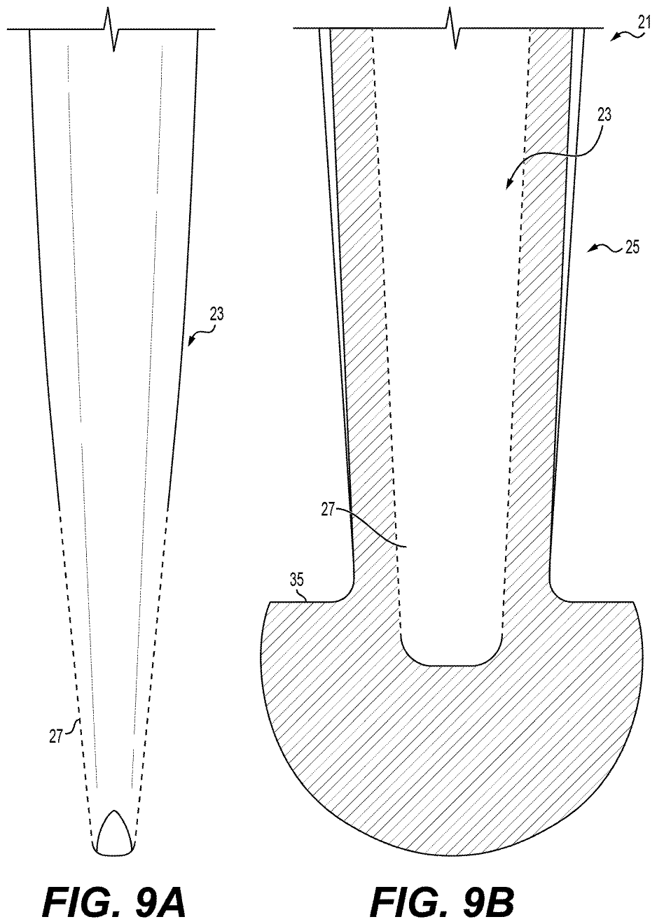

Referring to FIGS. 9A and 9B, the distal centralizer 25 is configured such that the distal component 35 engages the anatomical canal while permitting bone cement to flow around the outer surface thereof. The concave and expanding geometry of the distal component 35 facilitates insertion into a canal containing pressurized bone cement and assists in distributing the cement uniformly around the distal end 27. This configuration reduces the likelihood of direct contact between the hip stem 23 and cortical bone, thereby minimizing edge loading and improving implant stability.

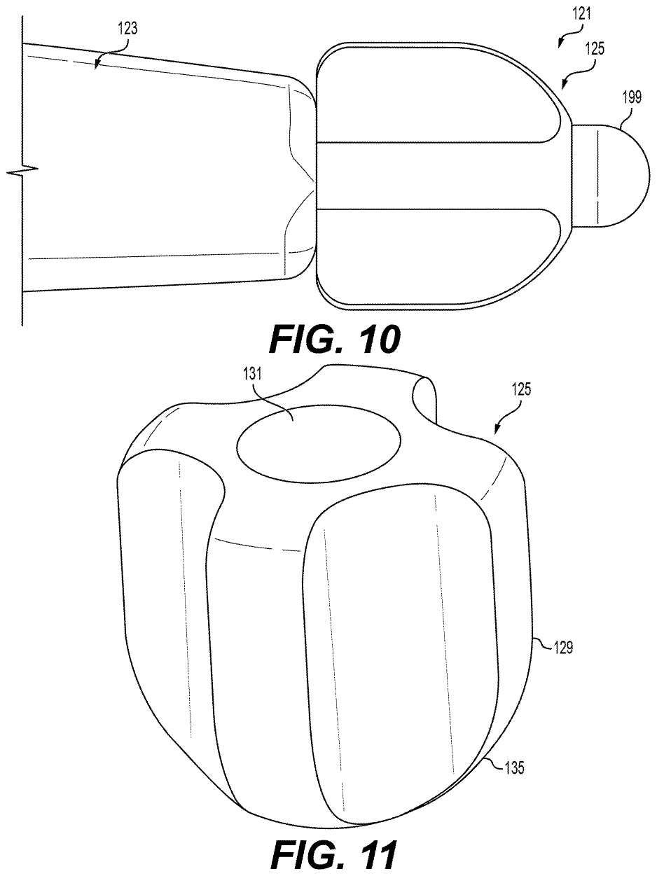

FIGS. 10-14 illustrate another potential implementation of the present disclosure. A hip implant system 121 has a distal centralizer 125 receivable on a hip stem 123. Distal centralizer 125 makes use of a cylindrical post 199. The cylindrical post 199 may have a substantially uniform diameter and length across multiple sizes of the hip stem 123, thereby providing a standardized attachment interface for the distal centralizer 125.

The distal centralizer 125 includes an internal cavity or bore 131 configured to receive the cylindrical post 199. The bore 131 is dimensioned to provide a frictional or interference fit with the cylindrical post 199, such that the distal centralizer 125 is retained on the hip stem 123 without the need for additional fixation elements. In some implementations, the fit between the cylindrical post 199 and the bore 131 is sufficient to allow the distal centralizer 125 to support its own weight when assembled with the hip stem 123.

The distal centralizer 125 further includes a distal portion 135 having an external surface 129 configured to engage an inner surface of the anatomical canal. The distal portion 135 may include a generally rounded, hemispherical, or otherwise smoothly contoured outer geometry to facilitate insertion into a cement-filled canal and to promote uniform distribution of bone cement around the distal centralizer 125. In certain implementations, the distal portion 135 permits bone cement to flow around the outer surface thereof.

In certain implementations, the distal portion 135 is configured such that an outer diameter of the distal centralizer 125 varies across different sizes of the distal centralizer 125, while an overall height or longitudinal length of the distal centralizer 125 remains substantially constant. This configuration enables the distal centralizer 125 to accommodate variations in canal diameter while maintaining a consistent positioning relative to the distal tip of the hip stem 123.

The cylindrical post 199 is preferably aligned with a longitudinal axis of the hip stem 123 and is centered about an anatomical canal axis such that offset compensation is not required. In this configuration, when the distal centralizer 125 is mounted on the cylindrical post 199, the distal centralizer 125 is concentrically aligned with the stem axis, thereby facilitating positioning of the hip stem 123 along the anatomical canal axis during implantation.

In use, the distal centralizer 125 is coupled to the cylindrical post 199 of the hip stem 123 prior to insertion of the hip stem 123 into a prepared femoral canal. The distal portion 135 of the distal centralizer 125 engages opposing regions of the canal or a cement mantle formed therein, thereby maintaining the hip stem 123 in a centralized position within the canal and reducing the likelihood of direct contact between the hip stem 123 and cortical bone.

The distal centralizer 125 may be formed from one or more biocompatible materials, including but not limited to polymethyl methacrylate (PMMA), polyether ether ketone (PEEK), or other polymeric materials compatible with bone cement and capable of withstanding thermal and mechanical conditions associated with cement curing.

The use of the cylindrical post 199 and corresponding bore 131 provides a modular and simplified interface between the hip stem 123 and the distal centralizer 125, thereby reducing manufacturing complexity and inventory requirements as compared to size-specific, geometry-matching centralizers. Additionally, the ability to vary the outer diameter of the distal portion 135 independently of the attachment interface enables customization to patient-specific canal geometries while maintaining a standardized coupling mechanism.

Now with reference to FIGS. 15, 16A, 16B, 16C, 17A, 17B, and 17C; in another potential implementation according to the present disclosure, hip implant system 221 makes use of a distal centralizer 225 receivable on hip stem 223. The distal centralizer 225 is configured to cooperate with the distal geometry of the hip stem 223 to facilitate alignment of the hip stem 223 within an anatomical canal C and to promote formation of a substantially uniform cement mantle M about the distal region of the hip stem 223.

The distal centralizer 225 includes a proximal portion 233 defining a mating interface 232 configured to receive and engage the distal portion of the hip stem 223. The mating interface 232 has a geometry that substantially corresponds to an external geometry of the distal portion of the hip stem 223, such that the distal centralizer 225 is conformingly receivable on the hip stem 223. In particular, the mating interface 232 is defined to match multi-axis taper characteristics of the hip stem 223, including medial/lateral (M/L), anterior/posterior (A/P), and proximal/distal taper profiles.

The mating interface 232 may be dimensioned to provide a frictional or interference fit with the distal portion of the hip stem 223, such that the distal centralizer 225 is retained on the hip stem 223 without the need for additional fastening components. In certain implementations, the engagement between the distal centralizer 225 and the hip stem 223 is sufficient to allow the distal centralizer 225 to remain secured to the hip stem 223 under its own weight prior to insertion into the anatomical canal.

In certain implementations, the distal centralizer 225 further includes a plug portion 240 associated with the proximal portion 233 and/or the mating interface 232. The plug portion 240 is configured to be received within a corresponding recess or opening defined in the distal portion of the hip stem 223, or within the mating interface 232, to enhance positional stability of the distal centralizer 225 relative to the hip stem 223.

The plug portion 240 may be integrally formed with the distal centralizer 225 or may be provided as a separate insert coupled thereto. The plug portion 240 may include a projection configured for frictional, interference, or snap-fit engagement with the corresponding recess of the hip stem 223, thereby improving retention of the distal centralizer 225 during handling and implantation.

In some implementations, the plug portion 240 is configured to at least partially occlude the mating interface 232 and/or a cavity associated with the distal portion of the hip stem 223, thereby limiting ingress of bone cement into the interface region. By restricting cement intrusion, the plug portion 240 helps maintain proper seating of the distal centralizer 225 on the hip stem 223 and prevents disruption of the conforming engagement between the mating interface 232 and the hip stem 223.

The distal centralizer 225 further includes a distal component 235 having an outer surface 229 configured to engage opposing regions of the anatomical canal. The outer surface 229 may define a generally arcuate profile, and in certain implementations may be asymmetric relative to a central longitudinal axis of the distal centralizer 225, such that the distal component 235 is adapted to conform to a broach-prepared cavity and to accommodate anatomical variations. In certain implementations, the outer surface defines a central outer wall axis that is offset from a plug aperture axis defined by the inner plug wall.

In certain implementations, a centerpoint associated with the outer surface 229 of the distal component 235 is offset relative to a local geometric center of the distal portion of the hip stem 223. The offset configuration is selected such that, when the distal centralizer 225 is coupled to the hip stem 223, the centerpoint is alignable with an anatomical canal axis. In this manner, the distal centralizer 225 compensates for geometric asymmetries of the hip stem 223, including lateral relief or non-uniform tapering, thereby promoting proper alignment of the hip stem 223 within the canal. In certain implementations, the plug portion 240 may limit ingress of bone cement into the mating interface 232 and provide primary retention of the distal centralizer 225 on the hip stem 223 during implantation.

The distal component 235 may further define a profile that increases in effective diameter in a proximal direction, enabling progressive engagement with the surrounding canal or cement mantle. In some implementations, the distal component 235 may include non-circular external geometries, including lobed, clover-shaped, or diamond-shaped configurations, configured to occupy space within a broach-formed cavity while permitting flow of bone cement around the distal centralizer 225. In certain implementations, the distal centralizer 225 is configured to fit within a broach-prepared cavity and may not contact cortical bone, thereby maintaining spacing between the hip stem 223 and surrounding bone while permitting formation of a cement mantle.

In certain implementations, the distal centralizer 225 is provided in a plurality of sizes corresponding to respective sizes of the hip stem 223, wherein the geometry of the mating interface 232 varies with stem size to maintain conformity with the distal stem geometry. The external geometry of the distal component 235 may also vary with size such that the distal centralizer 225 remains within a broach-prepared cavity while effectively centralizing the hip stem 223. In certain implementations, the distal centralizer 225 is configured to compensate for an offset between a local center of a distal tip of the hip stem 223 and an anatomical canal axis, the offset arising due to lateral relief of the hip stem 223, such that the distal component 235 positions the hip stem 223 in alignment with the anatomical canal axis.

In use, the distal centralizer 225 is assembled onto the distal portion of the hip stem 223 such that the mating interface 232 engages the stem geometry and the plug portion 240, where present, is received within a corresponding recess. The assembled hip stem 223 and distal centralizer 225 are inserted into a prepared femoral canal, and the distal component 235 engages the canal or surrounding cement to maintain spacing between the hip stem 223 and cortical bone, thereby reducing the likelihood of edge loading and promoting uniform cement distribution.

The distal centralizer 225 may be formed from one or more biocompatible materials, including but not limited to polymethyl methacrylate (PMMA), polyether ether ketone (PEEK), or other polymeric materials compatible with bone cement. The geometry and material properties of the distal centralizer 225 may be selected to withstand insertion forces and to facilitate controlled interaction with the surrounding cement and bone structures.

By providing a mating interface 232 that corresponds to the multi-axis taper geometry of the hip stem 223, in combination with the plug portion 240 and the offset distal component 235, the distal centralizer 225 enables stable attachment, controlled cement interaction, and improved alignment of the hip stem 223 with the anatomical canal axis, thereby enhancing implant positioning and long-term performance.

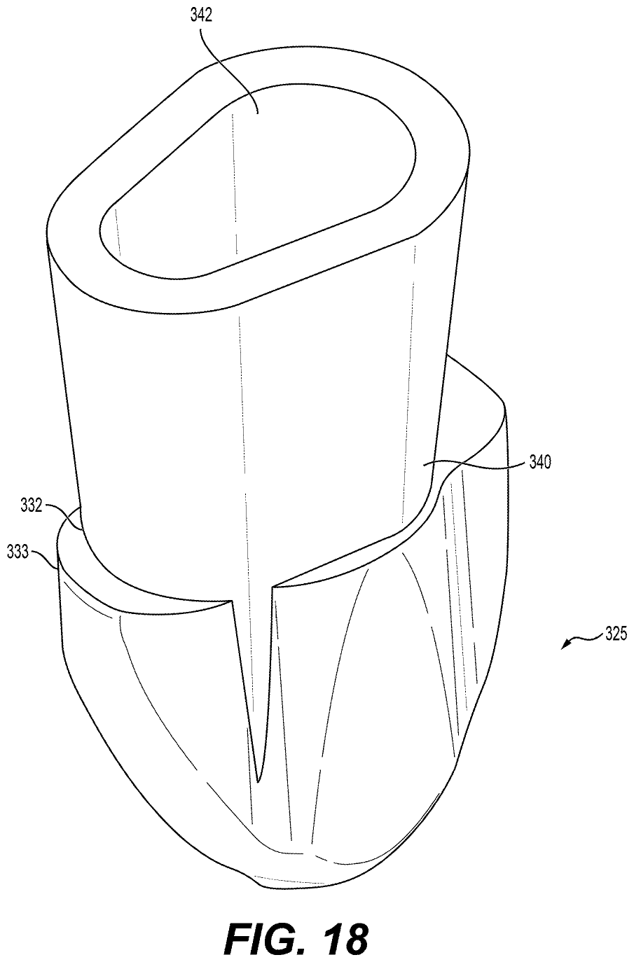

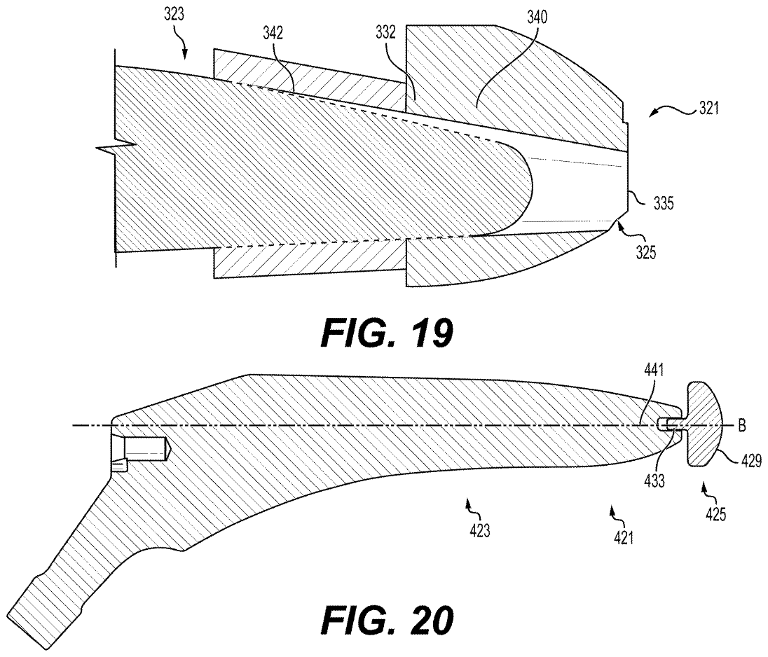

FIGS. 18-19 illustrate another implementation of the hip implant system 321 comprising a distal centralizer 325, receivable on a distal portion of a hip stem 323. In this embodiment, the distal centralizer 325 includes a proximal portion 333 having a mating interface 332 and a plug 340 configured to engage a corresponding recess of the hip stem 323. An inner plug wall 342 extends proximally from the plug 340 into the mating interface 332, as best shown in FIG. 19. The inner plug wall 342 defines at least a portion of the internal cavity and may be configured to conform to the geometry of the distal portion of the hip stem 323, thereby enhancing engagement between the distal centralizer 325 and the hip stem 323.

The proximally extending inner plug wall 342 may further function to improve retention and alignment of the distal centralizer 325 relative to the hip stem 323, including by providing additional surface contact and structural support within the mating interface 332. In certain implementations, the inner plug wall 342 cooperates with the plug 340 to limit ingress of bone cement into the mating interface 332, thereby maintaining proper seating of the distal centralizer 325 on the hip stem 323 during insertion. As illustrated in FIG. 19, the distal centralizer 325 is engaged with the hip stem 323 such that the plug 340 and inner plug wall 342 are received within the distal portion of the hip stem 323, while a distal portion 335 of the distal centralizer 325 is positioned to engage an anatomical canal to centralize the hip stem 323.

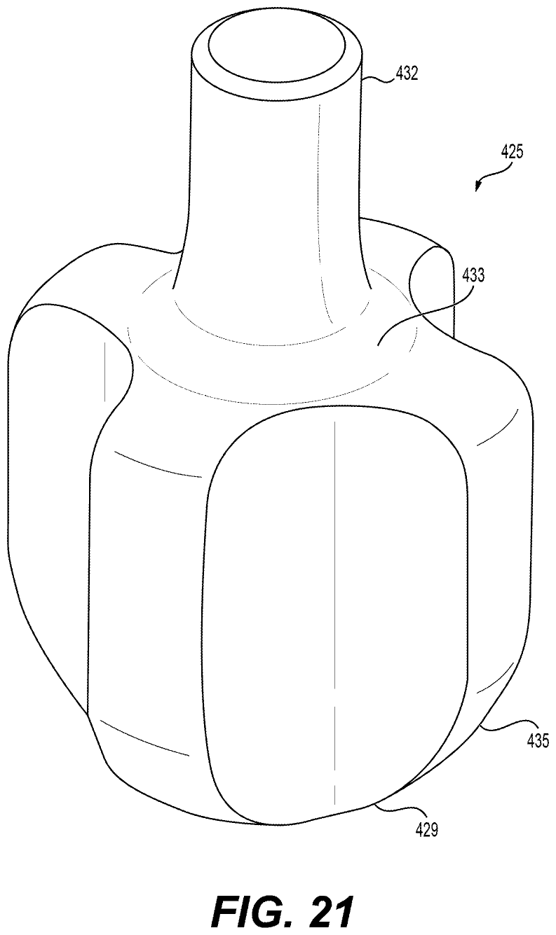

Yet another potential implementation according to the present disclosure is shown with reference to FIGS. 20-21, hip implant system 421 includes a hip stem 423 and a distal centralizer 425 receivable thereon. The distal end of the hip stem 423 terminates in portions defining a bore 441 extending longitudinally and terminating in a receiving aperture 443, the bore 441 having a central bore axis B aligned with an overall stem axis. The distal centralizer 425 includes a distal component 435 having an outer surface 429 defined by a concave profile, which extends arcuately about a centerpoint and is configured to engage opposing regions of an anatomical canal to centralize the hip stem 423 and promote formation of a uniform cement mantle.

The distal centralizer 425 further includes a proximal component 433 comprising a shank 432 extending proximally from the distal component 435, the shank 432 being sized to be received through the receiving aperture 443 and into the bore 441. The shank 432 defines a central shank axis that is medially offset from the central bore axis by an amount corresponding to a medial offset between a local stem axis and the overall stem axis of the hip stem 423. In this manner, upon insertion of the shank 432 into the bore 441, the distal centralizer 425 is positioned such that the concave outer surface 429 is aligned relative to the anatomical canal axis, thereby compensating for geometric asymmetry of the distal stem and facilitating proper alignment and stabilization of the hip stem 423 within the canal. In certain implementations, the shank is secured via an interference fit.

In certain implementations, the medial offset of the shank 432 corresponds to an offset between a local stem axis and an anatomical canal axis, such that insertion of the shank 432 into the bore 441 positions the hip stem 423 in alignment with the anatomical canal axis.

The operation and associated advantages of the various embodiments of hip implant systems and their associated hip stems and distal centralizers, are apparent from the foregoing description.

The distal centralizer will help align the overall stem axis with the anatomic canal axis and reduce potential drawbacks from misalignment. Such improved alignment may likewise contribute to creating a uniform cement mantle.

The disclosed features of the distal centralizer are configured to mate with unique or corresponding geometries at the distal end of the hip stem, such as tapering or other contours and surface features associated with the medial/lateral, anterior/posterior, and proximal/distal geometry, including tapering along three surfaces of cemented hip stems. The disclosed components are able to be used in the same broach system as current press fit stems and thus allow surgeons to preoperatively/intraoperatively decide whether to use a press-fit option without a distal centralizer or a cemented option with a suitable distal centralizer, including at a decision point after the surgeon has broached.

The disclosed hip implant systems have the further advantage that the associated distal centralizer will support their own weight during use, such as when placed on the hip stem and inserted in the canal whether natural or broached. This configuration will facilitate the surgeon's inserting the cemented hip stem.

As a still further advantage, the disclosed geometries of the centralizer distal feature are conducive to being pushed into a canal prepared with pressurized bone cement, and allows for cement to flow around.

As yet another advantage, when used in cemented stem applications, the disclosed geometries of the distal centralizer and corresponding distal ends allow for a uniform cement mantle when using the same broach system as the press fit stems. For example, the distal centralizer helps create a uniform cement mantle by preventing the stem from contacting the cortices, and otherwise optimizes placement of the stem within its broach cavity.

Additionally, the distal centralizer is easily attachable to the cemented stem and can retain its position and suitable form with minimal, if any, extra effort from the surgeon or operating room staff.

Among additional advantages, in the event that a surgeon prepares for a press fit stem, and later realizes that the bone quality is too poor to support a press fit stem, the surgeon can use the cemented stem and distal centralizer and may not need to further prepare the femur with broaching or reaming. This may enhance operating room efficiencies, as well as making the procedure more efficient and effective with regard to the patient's particular bone anatomy.

These and other advantages of the present disclosure will be apparent to those skilled in the art from the foregoing specification. Accordingly, it will be recognized by those skilled in the art that changes or modifications may be made to the above-described implementations without departing from the broad inventive concepts. It should therefore be understood that this disclosure is not limited to the particular implementations described herein, but is intended to include all changes and modifications that are within the scope and spirit of the disclosure as defined in the claims.

What is claimed is:

1. A hip implant system for use on a patient undergoing an arthroplasty procedure, the patient having an associated femur characterized by an anatomical axis, the system comprising:
a hip stem extending distally from a base to a distal end, the stem configured to be insertable into the associated femoral canal and to be operatively securable within the canal, the hip stem having an overall stem axis extending distally and alignable with the anatomical axis after insertion, the distal end having a local stem axis which is parallel to the overall stem axis, the local stem axis having a medial offset from the overall stem axis; and the distal centralizer securable to the distal end with an interference fit;
wherein the distal centralizer has first and second surfaces, the first surface adapted to engage the anatomical canal and defining a first, proximal-distal axis, the second surface adapted to engage the distal end and defining a second, proximal-distal axis;
wherein the first axis is laterally offset from the second axis by an amount corresponding to the medial offset between the local stem axis and the overall stem axis, whereby, upon attachment of the distal centralizer to the distal end, and upon insertion of the hip stem into the anatomical canal, the distal end is centralized to align the overall stem axis and the anatomical stem axis.

2. The system of claim 1, wherein the distal centralizer comprises proximal and distal components;
wherein the first surface is disposed on the distal component and has a concave profile with a centerpoint located to be alignable with the overall stem axis when the distal centralizer is attached to the hip stem, the first surface extending arcuately and symmetrically from the centerpoint to define respective increasing diameters at respective, proximally spaced locations on the distal component, one of the diameters engaging with opposing portions of the anatomical canal;
wherein the proximal component comprises a receptacle with a proximally oriented opening, the receptacle sized to be received on the distal end of the hip stem in an interference fit, the receptacle having an inner wall with a bottom point medially offset from the centerpoint by an amount corresponding to the medial offset between the local stem axis and the overall stem axis, the inner wall configured to mate with opposing surfaces of the distal end;
whereby the inner wall of the receptacle corresponds to the second surface of the distal centralizer.

3. The system of claim 1, wherein the distal centralizer comprises a cylindrical post secured to the distal end and having an outer cylindrical wall and a circumferential cross-section characterized by a central post axis extending longitudinally through the center of the circumferential cross-section;
wherein the cylindrical post extends distally from the distal end of the hip stem, the cylindrical post having a base secured to the distal end, the base having a central base axis extending distally and offset laterally from the local stem axis by an amount corresponding to the medial offset between the local stem axis and the overall stem axis;
wherein the distal centralizer further comprises a bushing having an inner bushing wall defining a bushing bore therethrough and sized to receive the cylindrical post therein in an interference fit;
wherein the bushing has a resiliently compressible, outer, concave surface oriented distally and having a profile extending symmetrically from the central base axis to define increasing diameters in the proximal direction;
wherein the base includes the first surface of the distal centralizer thereon; and
wherein the outer concave surface of the bushing corresponds to the second surface of the distal centralizer and thereby aligns the central base axis of the cylindrical post with the anatomical axis.

4. The system of claim 1, wherein the distal centralizer comprises a plug with inner and outer plug walls, the inner plug wall defining a plug aperture with a plug aperture axis alignable with the local stem axis when the plug is received on the hip stem, the inner plug wall corresponding to the first surface of the distal centralizer, the plug aperture axis corresponding to the first proximal-distal axis and the local stem axis, whereby the plug aperture axis is offset medially from the overall hip stem axis when the plug is received on the hip stem;
wherein the second surface of the digital centralizer is disposed on the outer plug wall to engage the anatomical canal;
wherein the outer plug wall is spaced asymmetrically from corresponding locations on the inner plug wall, the outer plug wall defining a central, outer plug wall axis offset from the inner plug wall axis by an amount corresponding to the medial offset of the local stem axis from the overall stem axis, whereby, when the plug is received on the distal end, the overall stem axis is aligned with the anatomical axis.

5. The system of claim 4, wherein the inner plug wall extends proximally from the plug.

6. The system of claim 1, wherein the distal end terminates in portions defining a bore extending longitudinally and terminating in a receiving aperture, the bore having a central bore axis aligned with the overall stem axis;
wherein the proximal component comprises a shank with a central shank axis, the shank extending proximally from the distal component, the shank sized to be received through the receiving aperture and into the bore; and
wherein the shank axis is medially offset from the central bore axis by an amount corresponding to the medial offset between the local stem axis and the overall stem axis.

7. The system of claim 2, wherein the inner wall of the receptacle includes medial/lateral taper surfaces corresponding to the distal end of the hip stem.

8. The system of claim 2, wherein the inner wall further includes anterior/posterior taper surfaces corresponding to the distal end of the hip stem.

9. The system of claim 2, wherein the distal component includes a non-circular outer geometry selected from a clover-shaped or diamond-shaped configuration configured to conform to a broach-prepared cavity.

10. The system of claim 3, wherein the cylindrical post has a uniform geometry across a plurality of hip stem sizes.

11. The system of claim 3, wherein the distal centralizer has a substantially constant height across different sizes while an outer diameter varies to accommodate anatomical canal sizes.

12. The system of claim 3, wherein the distal centralizer is configured to permit bone cement to flow around the outer concave surface during insertion into the anatomical canal.

13. The system of claim 4, wherein the outer plug wall defines a concave external surface configured to engage the anatomical canal.

14. The system of claim 4, wherein the outer plug wall is positioned asymmetrically relative to the inner plug wall such that a central outer wall axis is offset from a plug aperture axis.

15. The system of claim 5, wherein the inner plug wall extends proximally along the distal end of the hip stem to increase engagement length and retention.

16. The system of claim 5, wherein the plug is configured to limit ingress of bone cement into an interface between the distal centralizer and the hip stem.

17. The system of claim 6, wherein the distal component includes a concave outer surface extending arcuately and symmetrically from a centerpoint to define increasing diameters in a proximal direction.

18. The system of claim 6, wherein the shank is secured within the bore via an interference fit.

19. A hip implant system comprising:
a hip stem having a distal end defining medial/lateral, anterior/posterior, and proximal/distal taper geometries, the distal end having a local stem axis that is parallel to and offset from an overall stem axis; and
a distal centralizer comprising:
a proximal component including a receptacle having an inner wall configured to receive the distal end of the hip stem in an interference fit, the inner wall including surfaces corresponding to the taper geometries of the distal end; and
a distal component including an outer surface configured to engage an anatomical canal;
wherein the outer surface defines a concave profile extending arcuately and symmetrically from a centerpoint to define increasing diameters in a proximal direction; and
wherein the proximal component is positioned relative to the distal component such that the centerpoint is alignable with the overall stem axis to centralize the hip stem within the anatomical canal.

20. A hip implant system comprising:
a hip stem having a distal end with a local stem axis offset relative to an overall stem axis; and
a distal centralizer comprising:
a plug including an inner plug wall defining a plug aperture configured to receive the distal end of the hip stem, and an outer plug wall configured to engage an anatomical canal;
wherein the outer plug wall defines a concave external surface extending arcuately and symmetrically to define increasing diameters in a proximal direction;
wherein the outer plug wall is positioned asymmetrically relative to the inner plug wall such that a central axis of the outer plug wall is offset from an axis of the plug aperture by an amount corresponding to the offset between the local stem axis and the overall stem axis; and
wherein the distal centralizer is configured to align the hip stem with an anatomical canal axis.
